SEQUENCE LISTING (10 sequences)

SEQ ID NO: 1 - DNA, 531 bp, Homo sapiens
atgaagttacagtgtgtttccctttggctcctgggtacaatactgatattgtgctcagtagacaaccacggtctcaggagatgtctgatttccacagacatgcaccatatagaagagagtttccaagaaatcaaaagagccatccaagctaaggacaccttcccaaatgtcactatcctgtccacattggagactctgcagatcattaagcccttagatgtgtgctgcgtgaccaagaacctcctggcgttctacgtggacagggtgttcaaggatcatcaggagccaaaccccaaaatcttgagaaaaatcagcagcattgccaactctttcctctacatgcagaaaactctgcggcaatgtcaggaacagaggcagtgtcactgcaggcaggaagccaccaatgccaccagagtcatccatgacaactatgatcagctggaggtccacgctgctgccattaaatccctgggagagctcgacgtctttctagcctggattaataagaatcatgaagtaatgtcctcagct

SEQ ID NO: 2 - protein, 177 aa, Homo sapiens [sequence omitted]

SEQ ID NO: 3 - DNA, 594 bp, Homo sapiens
atggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattggagatatcgactacaaggatgacgacgacaagcacgtgcatcaccatcaccatcac

SEQ ID NO: 4 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 5 - DNA, 616 bp, Homo sapiens
atgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagc

SEQ ID NO: 6 - protein, 206 aa, Homo sapiens
MNFQQRLQSLWTLARPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 7 - DNA, 21 bp, Artificial Sequence
  Synthetic construct - primer
gagactgagagatgaattttc

SEQ ID NO: 8 - DNA, 24 bp, Artificial Sequence
  Synthetic construct - primer
gacattcagagcttgtagaatttc

SEQ ID NO: 9 - DNA, 22 bp, Artificial Sequence
  Synthetic construct - primer
caagtgagaggcatgaagttac

SEQ ID NO: 10 - DNA, 21 bp, Artificial Sequence
  Synthetic construct - primer
ccttgtcatcaagctgaggac

(19) United States
(12) Patent Application Publication
(10) Pub. No.: US 2005/0054567 A1
(43) Pub. Date: Mar. 10, 2005

(54) Use of il-19,il-22 and il-24 to treat hematopoietic disorders

(76) Inventor: Scott William Rowlinson, Indianapolis, IN (US)

Correspondence Address: ELI LILLY AND COMPANY;PATENT DIVISION, P.O. BOX 6288, INDIANAPOLIS, IN, 46206-6288, US

(21) Appl. No.: 10/491,995

(22) PCT Filed: Oct. 24, 2002
(86) PCT No.: PCT/US02/31599

Related U.S. Application Data
(60) Provisional application No. 60/332,986, filed on Nov. 6, 2001.

(51) Int. Cl. A61K 38/17

(52) U.S. Cl. 514/12

(57) ABSTRACT

The present invention relates to a method of using a mammalian gene sequence and polypeptides encoded thereby to treat mammalian hematopoietic disorders.

### FIELD OF THE INVENTION

The present invention relates to recombinant DNA technology as applied to the field of human medicine. In particular, the invention relates to methods of treating or preventing hematopoietic disorders that comprise the administration of IL-19, IL-22 or IL-24 to patients in need of such treatment.

### BACKGROUND OF THE INVENTION

Hematopoiesis is an essential, lifelong process whereby highly specialized blood cells are generated, including cells responsible for carbon dioxide and oxygen transport (erythrocytes), blood clotting (platelets), humoral immunity (B lymphocytes), cellular immunity (T lymphocytes), as well as cells which respond to foreign organisms and their products (granulocytes, monocytes, and macrophages). All of these cells can be functionally divided into two distinct groups termed myeloid and lymphoid. During normal adult life, myeloid cells are

produced exclusively within the bone marrow, while cells of the lymphoid lineage are produced to varying degrees in the bone marrow, spleen, thymus, and lymph nodes. Mature functional end cells and their immediate precursors have a limited life-span and a limited proliferative capacity and hence are not self-maintaining. Thus, these cells are continuously replaced from a pool of more primitive proliferating cells. Ultimately, all cells of both the myeloid and lymphoid lineage are derived from totipotent stem cells.

Hematopoiesis is necessarily tightly regulated. Hematopoietic cytokine action results in hematopoietic stem cell proliferation and differentiation, ultimately into the hematopoietic cells: red blood cells, platelets, granulocytes and monocytes. Development of a single cell type from a stem cell may require the coordinated action of a plurality of cytokines acting in the proper sequence.

Interleukin-20 (IL-20) is a recently described IL-10-related cytokine (International Patent Publications WO 99/27103 and WO 00/12708). The IL-20 coding sequence maps to human chromosome 1q32. This is the same region to which the genes encoding IL-10, IL-19 and IL-24 map, which are also IL-10-related cytokines. IL-20 has been described to have hematopoietic activity (U.S. Patent Application No. 60/272,242; Filed Feb. 28, 2001 incorporated herein by reference). IL-22 activates the JAK-STAT signaling pathway and modestly inhibits IL-14 production. Also, there are elevated IL-22 mRNA levels in stimulated T-cells. The biological activities of IL-24 and IL-19 are not well understood. U.S. Pat. No. 5,985,614 teaches the nucleotide and amino acid sequences encoding human IL-19. Reference 3, listed hereinbelow, teaches the nucleotide and amino acid sequences encoding IL-22.

Cytokine receptors are composed of one or more integral membrane proteins that bind a cytokine with high affinity and transduce the binding event into the cell through the cytoplasmic portions of the receptor subunits. Class II cytokine receptors, such as those that bind IL-19, IL-20, IL-22 and IL-24, are typically heterodimers composed of two distinct receptor chains, the α and β chains. The class II cytokine receptor subunits are not always exclusive to the binding of a single type of cytokine. For example, the IL-10 receptor complex is composed of IL-10Rα and IL-10Rβ, while IL-22 also uses IL-10Rβ in combination with IL-22R in its receptor complex. IL-20, IL-24 and IL-19 all have been shown to bind the receptor complex composed of IL-20Rα and IL-20Rβ (1). Both IL-24 and IL-20 also bind to a receptor complex composed of IL-22R and IL-20Rβ (1).

Hematopoietic cytokines have been successfully used in mammals to treat various diseases arising from imbalances between degradation and reconstitution of blood cells or from generation of inappropriate numbers of certain blood cells. For example, recombinant erythropoietin (EPO) is a glycoprotein administered for the treatment of anemia in chronic renal failure patients, zidovudine-treated HIV-infected patients, cancer patients on chemotherapy, and recently, patients receiving autologous transfusions. Recombinant thrombopoietin (TPO) is currently undergoing evaluation for treatment of thrombocytopenia, thrombopoiesis and anemia.

In spite of the availability of EPO and TPO, there remains a particular need to provide additional methods of altering the hematopoietic state of an individual. There is a particular need for proteins able to stimulate production of one, or more than one, type of hematopoietic cell. Accordingly, the present invention provides novel methods of treatment that can improve or prevent an undesired hematopoietic condition in a patient.

### SUMMARY OF THE INVENTION

The present invention provides a method for modulating hematopoiesis, including erythropoiesis (production of red blood cells), leukopoiesis (production of white blood cells) and/or thrombocytopoiesis (production of platelets) that comprises administering a therapeutically-effective amount of a pharmaceutical composition comprising, (alternatively, consisting of) at least one IL-19 agonist, IL-22 agonist, IL-24 agonist, IL-19 antagonist, IL-22 antagonist, IL-24 antagonist, IL-19 polypeptide, IL-22 polypeptide, IL-24 polypeptide or variant thereof, as defined herein, to a cell, tissue, organ, mammal, or patient, preferably a human, in need of such therapy to modulate hematopoietic activity.

One embodiment of the present invention is a method for using a therapeutically effective amount of at least one purified and isolated IL-19, IL-22, or IL-24 agonist, polypeptide or variant thereof to treat a mammal or patient, preferably a human, in need of a method for increasing hematopoietic cells.

Another embodiment of the present invention is a method for using a therapeutically effective amount of at least one purified and isolated IL-19, IL-22 or IL-24 antagonist to treat a mammal, human or patient in need of a method for decreasing hematopoietic cells.

### BRIEF DESCRIPTION OF THE FIGURES

FIG. 1 represents the nucleotide sequence encoding human IL-19 (SEQ ID No. 1).

FIG. 2 represents the amino acid sequence of human IL-19 with the signal sequence underlined (SEQ ID No. 2). Amino acids about 25-177 represent the mature form of IL-19.

FIG. 3 represents the nucleotide sequence encoding human IL-22 (SEQ ID No. 3).

FIG. 4 represents the amino acid sequence of human IL-22 with the signal sequence underlined (SEQ ID No. 4). Amino acids about 28-179 represent the mature form of IL-22.

FIG. 5 represents the nucleotide sequence encoding human IL-24 (SEQ ID No. 5).

FIG. 6 represents the amino acid sequence of human IL-24 with the signal sequence underlined (SEQ ID No. 6). Amino acids about 26-206 represent the mature form of IL-24.

### DETAILED DESCRIPTION OF THE INVENTION

The invention is not limited to the particular embodiments described below, as variations may be made and still fall within the scope of the appended claims. The terminology used herein is for the purpose of describing particular embodiments, and is not intended to be limiting. Instead, the scope of the present invention will be established by the appended claims.

Definitions

The term “amino acid” is used herein in its broadest sense, and includes naturally occurring amino acids as well as non-naturally occurring amino acids, including amino acid variants and derivatives. The latter includes molecules containing an amino acid moiety. Reference herein to an amino acid includes, for example, naturally occurring proteogenic L-amino acids; D-amino acids; chemically modified amino acids such as amino acid variants and derivatives; naturally occurring non-proteogenic amino acids such as norleucine, β-alanine, omithine, etc.; and chemically synthesized compounds having properties known in the art to be characteristic of amino acids.

The incorporation of non-natural amino acids, including synthetic non-native amino acids, substituted amino acids, or one or more D-amino acids into an IL-19 agonist, IL-22 agonist, IL-24 agonist, IL-19 antagonist, IL-22 antagonist, IL-24 antagonist, IL-19 polypeptide, IL-22 polypeptide, IL-24 polypeptide or a variant of IL-19 agonist, IL-22 agonist, IL-24 agonist, IL-19 antagonist, IL-22 antagonist, IL-24 antagonist, IL-19 polypeptide, IL-22 polypeptide, IL-24 polypeptide (collectively referred to herein as “polypeptides of the invention”) is advantageous in that they exhibit increased stability in vitro or in vivo compared to L-amino acid-containing counterparts. D-peptides are resistant to endogenous peptidases and proteases, thereby providing improved bioavailability of the molecule, and prolonged lifetimes in vivo. When it is desirable to allow the polypeptide of the invention, to remain active for only a short period of time, the use of L-amino acids therein will permit endogenous peptidases, proteases, etc., to digest the molecule, thereby limiting the cells exposure to the molecule. Additionally, D-amino acid containing polypeptides, cannot be processed efficiently for major histocompatibility complex class II-restricted presentation to T-helper cells, and are less likely to induce humoral immune responses in a whole organism.

In addition to using D-amino acids, those of ordinary skill in the art are aware that modifications in the amino acid sequence of an IL-19, IL-22 or IL-24 polypeptide can result in functional polypeptides that display equivalent or superior functional characteristics when compared to that of a protein having the original polypeptide sequence as shown in SEQ ID NOS: 2, 4 and 6 respectively. Thus, the methods of the present invention contemplate alterations in IL-19, IL-22 or IL-24 polypeptides or variants thereof that may include one or more amino acid insertions, deletions, substitutions, truncations, fusions, shuffling of subunit sequences, and the like, either from natural mutations or from artificial manipulation, provided that the sequences produced by such modifications have substantially the same (or improved or reduced, as may be desirable) activity(ies) as the unmodified polypeptides. Most preferably a modified polypeptide for use in the method of the invention has a sequence that is at least about 95% homologous to SEQ ID NOS: 2, 4 or 6, (preferably to the mature form of the polypeptide without the signal sequence). Even more preferably a modified polypeptide for use in the method of the invention has a sequence that is at least about 96%, 97%, 98%, or 99% homologous to SEQ ID NOS: 2, 4 or 6 (preferably to the mature form of the polypeptide without the signal sequence).

The term “antagonist” is used in the broadest sense and includes any molecule that partially or fully blocks, inhibits, or neutralizes a biological activity of a polypeptide of interest. In a similar manner, the term “agonist” is used in the broadest sense and includes any molecule that induces or increases the expression, stability, and/or biological activity of a polynucleotide or polypeptide of interest.

The term “functional” in reference to an IL-19, IL-22, or IL-24 polypeptide or variant thereof is intended to mean that the particular molecule exhibits biological activities, in vivo or in vitro, that are similar or identical to, or better than, the biological activities attributable to the IL-19, IL-22, or IL-24 polypeptides (with sequence shown in SEQ ID NOS: 2, 4, 6 respectively), as disclosed herein i.e., the ability to induce the growth and/or differentiation of hematopoietic progenitor cells.

The term “hematocrit” refers to a measurement of the ratio of the volume of red blood cells to the volume of whole blood cells as determined by any instrument used in determining the relative amounts of plasma and corpuscles in blood. It is understood that hematocrit targets will vary from one individual to another such that physician discretion may be appropriate in determining an actual target hematocrit for any given patient. Nonetheless, determining a target hematocrit is well within the level of skill in the art.

The term “IL-19 composition” refers to a composition of matter comprising of, or alternatively, consisting at least one IL-19 agonist, IL-19 antagonist, IL-19 polypeptide, IL-19 variant as defined herein and useful in the method of the invention. Likewise, the term “IL-22 composition” refers to a composition of matter comprising of, or alternatively, consisting of at least one IL-22 agonist, IL-22 antagonist, IL-22 polypeptide, IL-22 variant useful in the method of the invention. Likewise, “IL-24 composition” refers to a composition of matter comprising of, or alternatively, consisting of at least one IL-24 agonist, IL-24 antagonist, IL-24 polypeptide, IL-24 variant for use in the method of the invention. The IL-19, IL-22 and IL-24 polypeptides in compositions of the invention are preferably the mature form of the proteins, or variants thereof, without the signal sequence.

The term “IL-19 variant” as used herein refers to an IL-19 polypeptide (whose sequence is shown in SEQ ID NO: 2) that further comprises at least one of the various types of modifications contemplated herein. Furthermore, IL-19 variant, as applied to a polypeptide, is intended to refer to a “functional” IL-19 polypeptide, as defined herein, having at least about 95% amino acid sequence identity with an IL-19 polypeptide having the deduced amino acid sequences as shown in SEQ ID NO: 2 (with or without the signal peptide). Such IL-19 polypeptide variants include, for instance, IL-19 polypeptides wherein one or more amino acid residues are added, substituted or deleted, at the N- or C-terminus or within the sequence of SEQ ID NO: 2.

The term “IL-22 variant” as used herein refers to an IL-22 polypeptide (whose sequence is shown in SEQ ID NO: 4) that further comprises at least one of the various types of modifications contemplated herein. Furthermore, IL-22 variant, as applied to a polypeptide, is intended to refer to a “functional” IL-22 polypeptide, as defined herein, having at least about 95% amino acid sequence identity with an IL-22 polypeptide having the deduced amino acid sequence shown in SEQ ID NO: 4 (with or without the signal peptide). Such IL-22 polypeptide variants include, for instance, IL-22 polypeptides wherein one or more amino acid residues are added, substituted or deleted, at the N- or C-terminus or within the sequence of SEQ ID NO: 4.

The term “IL-24 variant” as used herein refers to a IL-24 polypeptide (whose sequence is shown in SEQ ID NO: 6) that further comprises at least one of the various types of modifications contemplated herein. Furthermore, IL-24 variant, as applied to a polypeptide, is intended to refer to a “functional” IL-24 polypeptide, as defined herein, having at least about 95% amino acid sequence identity with an IL-24 polypeptide having the deduced amino acid sequence shown in SEQ ID NO: 6 (with or without the signal peptide). Such IL-24 polypeptide variants include, for instance, IL-24 polypeptides wherein one or more amino acid residues are added, substituted or deleted, at the N- or C-terminus or within the sequence of SEQ ID NO: 6.

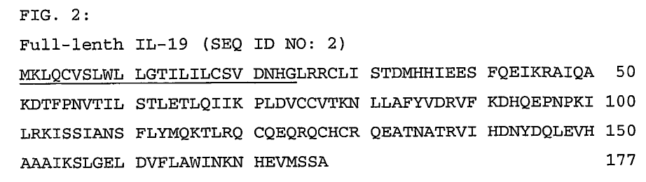

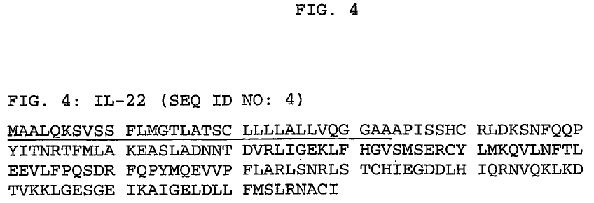

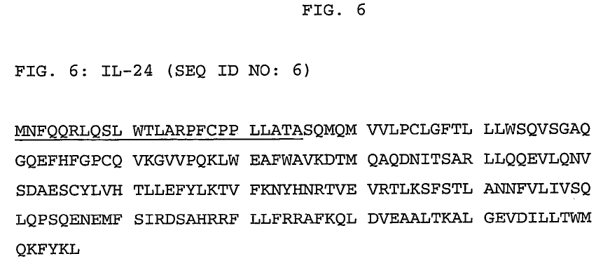

More preferably the variant polypeptides for use in the compositions and methods of the invention have at least about 96% amino acid sequence identity, even more preferably at least 97%, 98%, or 99% identity with the sequence represented in SEQ ID NOS: 2, 4 or 6 (FIGS. 2, 4, and 6), with or without the signal peptide, but preferably without.

The term “inhibit” or “inhibiting” includes the generally accepted meaning prohibiting, preventing, restraining, slowing, stopping, or reversing progression or severity of a disease or condition.

The term “mature protein” or “mature polypeptide” as used herein refers to the form(s) of a protein produced by expression in a mammalian cell. It is generally hypothesized that once export of a growing protein chain across the rough endoplasmic reticulum has been initiated, proteins secreted by mammalian cells have a signal peptide (SP) sequence which is cleaved from the complete polypeptide to produce a “mature” form of the protein. Oftentimes, cleavage of a secreted protein is not uniform and may result in more than one species of mature protein. The cleavage site of a secreted protein is determined by the primary amino acid sequence of the complete protein and generally cannot be predicted with complete accuracy. Methods for predicting whether a protein has a SP sequence, as well as the cleavage point for that sequence, are available. A cleavage point may exist within the N-terminal domain of IL-19, IL-22 or IL-24 between amino acid 10 and amino acid 35. The predicted cleavage sites for IL-19, IL-22 and IL-24 signal peptide are shown in FIGS. 2, 4 and 6. The actual cleavage site may vary by up to about 6 amino acids on either side of the predicted cleavage site. As one of ordinary skill would appreciate, cleavage sites sometimes vary from organism to organism and cannot be predicted with absolute certainty. Optimally, cleavage sites for a secreted protein are determined experimentally by amino-terminal sequencing of the one or more species of mature proteins found within a purified preparation of the protein.

The term “recombinant DNA expression vector” or “expression vector” as used herein refers to any recombinant DNA cloning vector, for example a plasmid or phage, in which a promoter and other regulatory elements are present thereby enabling transcription of an operably linked DNA, which may encode a protein.

The term “isolated” when used in relation to a nucleic acid or protein, means the material is identified and separated from at least one contaminant with which it is ordinarily associated in its natural source. Such a nucleic acid could be part of a vector and/or such nucleic acid or protein could be part of a composition, and still be isolated in that such vector or composition is not part of its natural environment.

As used herein, the term “purified” means the result of any process that removes from a sample a contaminant from the component of interest, such as a protein or nucleic acid. The percent of a purified component is thereby increased in the sample.

The term “increasing hematopoietic cells” is used herein to denote the restoration or enhanced recovery of hematopoietic cells following their ablation such as ablation resulting from disease, disorder or therapeutic intervention.

The term “treatment” or “treating” as used herein describes the management and care of a patient for the purpose of combating or preventing a disease, condition, or disorder and includes the administration of at least one IL-19, IL-22 or IL-24 agonist, IL-19, IL-22 or IL-24 antagonist, isolated and purified IL-19, IL-22 or IL-24 polypeptide or variant thereof, or a composition comprising (or alternatively, consisting of or essentially consisting of) IL-19, IL-22 or IL-24 agonist, IL-19, IL-22 or IL-24 antagonist, isolated and purified IL-19, IL-22 or IL-24 polypeptide or variant thereof to prevent the onset of the symptoms or complications, alleviating the symptoms or complications, or eliminating the disease, condition, or disorder as further defined herein for a mammal, human or patient in need of such treatment. An example of “preventive therapy” is the prevention or lessened targeted pathological condition or disorder. Those in need of treatment include those already with the disorder or disease as well as those prone to have the disorder or disease or those in whom the disorder or disease is to be prevented.

“Active” or “activity” in the context of variants of the polypeptides of the invention refers to retention of a biologic function of the polypeptide of the invention and/or the ability to bind to a receptor or ligand much as would an unmodified polypeptide of the invention. More specifically, “biological activity” refers to a biological function (either inhibitory or stimulatory) caused by a reference polypeptide. Exemplary biological activities include, but are not limited to, the ability of such molecules to induce or inhibit infiltration of inflammatory cells (e.g., leukocytes) into a tissue, to induce or inhibit adherence of a leukocyte to an endothelial or epithelial cell, to stimulate or inhibit T-cell proliferation or activation, to stimulate or inhibit cytokine release by cells or to increase or decrease vascular permeability.

Administration “in combination with” one or more further therapeutic agents includes simultaneous (concurrent) and consecutive administration in any order.

A “therapeutically-effective amount” is the minimal amount of active agent (e.g., an LP polypeptide) necessary to impart therapeutic benefit to a mammal, i.e., an amount that induces, ameliorates or otherwise causes an improvement in the pathological symptoms, disease progression, physiological conditions associated with or resistance to succumbing to the aforedescribed disorder.

Overview

It has been shown that upon exposure to IL-20 polypeptides in combination with human EPO and human stem cell factor, human CD34+ progenitor cells proliferate and differentiate markedly. Also, addition of IL-20 polypeptides greatly increased the sizes of erythroid progenitors (see U.S. Patent Application No. 60/272,242; Filed Feb. 28, 2001, incorporated herein). Here, applicants teach that isolated and purified polypeptides of the invention (mature IL-19 polypeptide, IL-22 polypeptide, IL-24 polypeptide and variants thereof) demonstrate the ability to stimulate proliferation and differentiation of a hematopoietic progenitor cell. A preferred embodiment of the invention is a method of using at least one isolated and purified mature IL-19, IL-22 or IL-24 polypeptide or variant thereof for the purpose of stimulating proliferation of greater than one type of hematopoietic progenitor cell (e.g., a progenitor of red blood cells and a progenitor of platelets). Another embodiment of the invention is a method of using at least one IL-19, IL-22 or IL-24 agonist for the purpose of stimulating proliferation of greater than one type of hematopoietic progenitor cell (e.g., a progenitor of red blood cells and a progenitor of platelets).

Further described herein is activity of IL-19, IL-22 and IL-24 closely associated with hematopoietic processes. Therefore, the present invention provides methods of treating or preventing hematopoietic disorders including, but not limited to, the administration of a therapeutically effective amount of a pharmaceutical composition comprising of, or alternatively consisting of or consisting essentially of, at least one IL-19, IL-22 or IL-24 agonist, IL-19, IL-22 or IL-24 polypeptide or variant thereof to a mammal, human or patient in need of such treatment to increase hematopoietic cells. Such methods are useful for enhancing or stimulating hematopoiesis, erythropoiesis, leukopoiesis, thrombocytopoiesis, production of neutrophils, granulocytes, and/or platelets by stimulating the proliferation and/or differentiation of progenitors of such cells, as needed in various conditions and/or situations, including, but not limited to, the following:
(a) inadequate platelet production, such as aplastic anemia, refractory anemias, leukemia, preleukemia/myelodysplastic syndromes, megaloblastic anemia, chemotherapy or radiation therapy, and existing platelet deficiency or an expected platelet deficiency (e.g., because of planned surgery including, but not limited to, organ/bone marrow transplantations);
(b) increased destruction of platelets, such as idiopathic thrombocytopenia purpura, other immune thrombocytopenias, IV-associated thrombocytopenia, sepsis/disseminated intravascular coagulation, and vasculitis;
(c) abnormal platelet function, such as Glanzmann's thrombasthenia, acute/chronic leukemia, myeloproliferative disorders, uremia, platelet storage pool disease, Von Willebrand disease, and postoperative cardiovascular dysfunction, and
(d) other blood coagulation disorders such as afibrinogenemia or wounds of any origin.

The generic term for platelet deficiency is thrombocytopenia, and hence the methods and compositions of the present invention are generally available for treating thrombocytopenias. Thrombocytopenias (platelet deficiencies) may be present for various reasons, including chemotherapy, radiation therapy, surgery, accidental blood loss, and other specific disease conditions. Exemplary specific disease conditions that involve thrombocytopenia and may be treated in accordance with this invention are: aplastic anemia, idiopathic thrombocytopenia, and certain metastatic tumors which result in thrombocytopenia. Also, certain treatments for AIDS result in thrombocytopenia (e.g., AZT). Certain wound healing disorders might also benefit from an increase in platelet numbers.

With regard to anticipated platelet deficiencies, (e.g., due to future surgery), at least one isolated and purified IL-19, IL-22 or IL-24 agonist, polypeptide, or variant thereof, could be administered several days to several hours prior to the need for platelets. With regard to acute situations, e.g., accidental and massive blood loss, at least one IL-19, IL-22 or IL-24 agonist, polypeptide, or variant thereof, could be administered along with blood or purified platelets or other cytokines, further described herein.

The present invention also provides methods of treating or preventing hematopoietic disorders including, but not limited to, the administration of a therapeutically effective amount of a pharmaceutical composition comprising of, or alternatively consisting of or consisting essentially of, at least one IL-19, IL-22 or IL-24 antagonist to a mammal, human or patient in need of such treatment to decrease hematopoietic cells.

Furthermore, the present invention provides an IL-19, IL-22 or IL-24 agonist, antagonist, polypeptide or variant thereof, and compositions comprising, or alternatively consisting of at least one IL-19, IL-22 or IL-24 agonist, antagonist, polypeptide or variant thereof that modulate intracellular signaling pathways dependent on at least one of the following: hematopoietic, erythropoietic, leukopoietic or thrombopoietic related function. IL-19, IL-22 or IL-24 molecules (i.e., agonists, antagonists, polypeptides and variants thereof) and/or IL-19, IL-22 or IL-24 compositions can stimulate (agonists, polypeptides and variants thereof) or inhibit (antagonists) T-cell activation and/or proliferation and, thereby, have therapeutic utility for treating infections caused by viruses including, but not limited to, HIV and have therapeutic utility for treating various autoimmune diseases including, but not limited to, rheumatoid arthritis, lupus, graft versus host, host versus graft, insulin-dependent diabetes, autoimmune encephlo-myelitis, and multiple sclerosis.

A preferred embodiment of the present invention provides methods of treating or preventing hematopoietic disorders including, but not limited to, anemia and disorders commonly associated with anemia comprising the administration to a mammal, human, or patient in need of such treatment a therapeutically effective amount of a pharmaceutical composition comprising an isolated IL-19, IL-22 or IL-24 agonist, polypeptide, or variant thereof, comprising (alternatively consisting of, alternatively consisting essentially of) at least one an IL-19 agonist, IL-22 agonist, IL-24 agonist, IL-19 polypeptide, IL-22 polypeptide, IL-24 polypeptide or a variant thereof.

A preferred embodiment of the present invention also provides methods of treating or preventing hematopoietic disorders such as anemia and/or disorders associated with anemia comprising the administration of a therapeutically effective amount of a pharmaceutical composition comprising (alternatively consisting of or consisting essentially of) an IL-19, IL-22 or IL-24 agonist, polypeptide or variant thereof as defined herein to a mammal or patient, preferably a human, in need of such treatment.

A preferred embodiment of the present invention provides a method of treating or preventing hematopoietic disorders including, but not limited to, erythrocytosis, and/or leukemia comprising the administration of a therapeutically effective amount of a pharmaceutical composition comprising a IL-19, IL-22 or IL-24 antagonist to a mammal, preferably a human, in need of such treatment.

The present invention further provides a pharmaceutical formulation that comprises (alternatively consists of or consists essentially of (i.e., less than about 10% impurities)) at least one IL-19, IL-22 or IL-24 agonist, IL-19, IL-22 or IL-24 antagonist, IL-19, IL-22 or IL-24 polypeptide or variant thereof and/or IL-19, IL-22 or IL-24 composition together with one or more pharmaceutically acceptable diluents, carriers, or excipients therefor.

The present invention also provides a method of treating or preventing hematopoietic disorders including, but not limited to, anemia and/or disorders commonly associated with anemia, comprising the administration to a mammal, human or patient in need thereof of a therapeutically effective amount of an IL-19, IL-22 or IL-24 composition wherein said composition has at least one activity, such as, but not limited to, inducing differentiation and/or proliferation of erythroid and/or megakaryocyte progenitor cells. An IL-19, IL-22 or IL-24 polypeptide can be screened for a corresponding activity according to these effects.

The invention further provides for the use of at least one IL-19, IL-22 or IL-24 agonist, IL-19, IL-22 or IL-24 antagonist, IL-19, IL-22 or IL-24 polypeptide or variant thereof, in the manufacture of a medicament for the treatment or prevention of anemia, leukemia and disorders associated with such conditions.

Variants

Reference to a particular polypeptide sequence disclosed in SEQ ID NOS: 2, 4 or 6, with or without the signal peptide sequence as shown in FIGS. 2, 4 and 6 (i.e., a “polypeptide of the invention”) as well as fusion proteins comprising polypeptides of the invention is also understood to include variants of the polypeptide as defined herein. The term “variant” refers to a polypeptide differing from a polypeptide of the present invention, but retaining essential properties thereof. Generally, variants are closely similar overall in structural and/or sequence identity, and, in many regions, identical to polypeptide of the present invention.

The present invention is also directed to polypeptides that comprise, or alternatively consist of, an amino acid sequence that is at least: 95%, 96%, 97%, 98%, 99% identical to a polypeptide sequence of SEQ ID NOs: 2, 4 or 6 (with or without the signal peptide sequence as depicted in FIGS. 2, 4 and 6).

A polypeptide exhibiting or having at least about, e.g., 95% “sequence identity” to another amino acid sequence may include, e.g., up to five amino acid alterations per each 100 amino acid (on average) stretch of the test amino acid sequence. In other words, a first amino acid sequence that is at least 95% identical to a second amino acid sequence, can have up to 5% of its total number of amino acid residues different from the second sequence, e.g., by insertion, deletion, or substitution of an amino acid residue.

Alterations in amino residues of a polypeptide sequence may occur at the amino or carboxy terminal positions or anywhere between these terminal positions, interspersed either individually among residues in the sequence or in one or more contiguous fragments within the sequence. As a practical matter, whether any particular polypeptide sequence exhibits at least about: 90%, 91%, 92%, 93%, 94% 95%, 96%, 97%, 98%, or 99% similarity to another sequence, can be determined conventionally by using known methods in the art.

The phrase “percent (%) identity” with respect to the amino acid sequences identified herein is defined as the percentage of amino acid residues in a candidate sequence that are identical with the amino acid residues in a reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as ALIGN, ALIGN-2, Megalign (DNASTAR) or BLAST (e.g., Blast, Blast-2, WU-Blast-2) software. Those skilled in the art can determine appropriate parameters for measuring alignment, including any algorithms needed to achieve maximal alignment over the full length of the sequences being compared. For example, the % identity values used herein are generated using WU-BLAST-2 [Altschul, et al., Methods in Enzymology 266: 460-80 (1996)]. Most of the WU-BLAST-2 search parameters are set to the default values. Those not set to default values, i.e., the adjustable parameters, are set with the following values: overlap span=1; overlap fraction=0.125; word threshold (T)=11; and scoring matrix=BLOSUM 62. For purposes herein, a % amino acid sequence identity value is determined by dividing (a) the number of matching identical amino acid residues between the amino acid sequence of the hSEZ6 polypeptide of interest and the comparison amino acid sequence of interest (i.e., the sequence against which the hSEZ6 polypeptide of interest is being compared) as determined by WU-BLAST-2, by (b) the total number of amino acid residues of the polypeptide of interest.

Variants may be produced by mutagenesis techniques or by direct synthesis using known methods of protein engineering and recombinant DNA technology. Such variants may be generated to improve or alter the characteristics of the polypeptide or the expression levels or may occur unintentionally. One or more amino acids can often be deleted from the N-terminus or C-terminus of a secreted polypeptide without a substantial loss of biological function. Moreover, ample evidence demonstrates that polypeptide variants can retain a biological activity similar to that of the naturally occurring protein. Even if deleting one or more amino acids from the N-terminus or C-terminus of the polypeptide results in modification or loss of one or more biological functions, other biological activities may be retained.

Variants of the polypeptides of the invention can be generated through DNA shuffling as disclosed, for example, by International Patent Application WO 97/20078 and U.S. Pat. Nos. 6,303,344 and 6,297,053.

The invention also encompasses polypeptide variants that show a biological activity of the reference polypeptide such as, e.g., ligand binding or antigenicity. Such variants include, e.g., deletions, insertions, inversions, repeats, and substitutions selected so as to have little effect on activity using general rules known in the art.

One technique compares amino acid sequences in different species to identify the positions of conserved amino acid residues since changes in an amino acid at these positions are more likely to affect a protein function. In contrast, the positions of residues where substitutions exist more frequent generally indicate that amino acid residues at these positions are less critical for a protein function. Thus positions tolerating amino acid substitutions typically may be modified while still maintaining a biological activity of a protein.

Another technique uses genetic engineering to introduce amino acid changes at specific positions of a polypeptide to identify regions critical for a protein function. For example, site directed mutagenesis or alanine-scanning mutagenesis (the introduction of single alanine mutations at every residue in the molecule) can be used. A resulting mutant can subsequently be tested for a biological activity.

These two techniques have revealed that proteins are surprisingly tolerant of amino acid substitutions and they generally indicate which amino acid changes are likely to be permissive at certain amino acid positions in a protein. For example, typically, most buried amino acid residues (those within the tertiary structure of the protein) require nonpolar side chains, whereas few features of surface side chains are generally conserved. Preferred conservative amino acid substitutions are listed in Table 1 herein.

Polypeptide variants containing amino acid substitutions of charged amino acids with other charged or neutral amino acids may produce polypeptides with improved characteristics e.g., such as less aggregation. Aggregation of pharmaceutical formulations both reduces activity and increases clearance due to the aggregate's immunogenic activity.

A further embodiment of the invention encompasses a protein that comprises an amino acid sequence of the present invention that contains at least one amino acid substitution, but not more than 15 amino acid substitutions, preferably not more than 10 amino acid substitutions.

Of course, in order of ever-increasing preference, it is highly preferable for a polypeptide of the invention to have an amino acid sequence that comprises an amino acid sequence of the present invention which contains zero or one, but not more than: 10, 9, 8, 7, 6, 5, 4, 3, 2, or 1 amino acid substitutions; wherein conservative amino acid substitutions are more preferable than non-conservative substitutions.

The novel methods contemplated by the present invention are intended to include methods of using IL-19, IL-22 or IL-24 polypeptides with a sequence as shown in SEQ ID NOS: 2, 4, and 6, respectively (preferably without the signal sequence), as well as active polypeptide variants thereof that further comprise one or more substitutions, deletions, insertions, inversions, additions yet have substantially similar or better biological activities and/or pharmaceutically desired properties as the corresponding unmodified IL-19, IL-22 or IL-24 polypeptide.

In one embodiment of the present invention, a single amino acid change is made within the IL-19, IL-22 or IL-24 polypeptide that has a sequence as shown in SEQ ID NOs: 2, 4 or 6 respectively (with or without the signal sequence). Alternatively, at least two changes are made within at least one of these polypeptide sequences; alternatively, at least three changes are made within at least one of these polypeptide sequences; alternatively, at least four changes up to at least 10 changes are made within at least one of these polypeptide sequences. As the skilled artisan understands, many substitutions, and/or other changes to a protein's sequence or structure, can be made without substantially affecting the biological activity or characteristics of the polypeptide. For example, making conservative amino acid substitutions, or changing one amino acid for another from the same class of amino acids, for example, negatively charged residues, positively charged residues, polar uncharged residues, and non-polar residues, or any other classification acceptable in the art, are often made without significant effects upon function. Modifications of the IL-19, IL-22 or IL-24 polypeptide with a sequence as shown in SEQ ID NO: 2, 4 or 6 respectively (with or without the signal sequence) made in accordance with Table I hereinbelow are expected to result in variant polypeptides that retain the same or substantially similar or even better biological activity as the unmodified polypeptide based on art recognized substitutability of certain amino acids and are also contemplated as being useful in the methods of the present invention.

TABLE I

| ORIGINAL RESIDUE | EXEMPLARY SUBSTITUTIONS |
|---|---|
| ALA | SER, THR |
| ARG | LYS |
| ASN | HIS, SER |
| ASP | GLU, ASN |
| CYS | SER |
| GLN | ASN, HIS |
| GLU | ASP, GLU |
| GLY | ALA, SER |
| HIS | ASN, GLN |
| ILE | LEU, VAL, THR |
| LEU | ILE, VAL |
| LYS | ARG, GLN, GLU, THR |
| MET | LEU, ILE, VAL |
| PHE | LEU, TYR |
| SER | THR, ALA, ASN |
| THR | SER, ALA |
| TRP | ARG, SER |
| TYR | PHE |
| VAL | ILE, LEU, ALA |
| PRO | ALA |

One factor that can be considered in making such changes is the hydropathic index of amino acids. The importance of the hydropathic amino acid index in conferring interactive biological function on a protein has been discussed by Kyte and Doolittle (2). It is accepted that the relative hydropathic character of amino acids contributes to the secondary structure of the resultant protein. This, in turn, affects the interaction of the protein with molecules such as enzymes, substrates, receptors, ligands, DNA, antibodies, antigens, etc. Based on its hydrophobicity and charge characteristics, each amino acid has been assigned a hydropathic index as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

As is known in the art, certain amino acids in a peptide, polypeptide, or protein can be substituted for other amino acids having a similar hydropathic index or score and produce a resultant peptide, etc., having similar biological activity, i.e., which still retains biological functionality. In making such changes, it is preferable that amino acids having hydropathic indices within ±2 are substituted for one another. More preferred substitutions are those wherein the amino acids have hydropathic indices within ±1. Most preferred substitutions are those wherein the amino acids have hydropathic indices within ±0.5.

Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5±1); alanine/histidine (−0.5); cysteine (−1.0); methionine (−1.3); valine (−1.5); leucine/isoleucine (1.8); tyrosine (−2.3); phenylalanine (−2.5); and tryptophan (−3.4). Thus, one amino acid in a peptide, polypeptide, or protein can be substituted by another amino acid having a similar hydrophilicity score and still produce a resultant peptide, etc., having similar biological activity, i.e., still retaining correct biological function. In making such changes, amino acids having hydropathic indices within ±2 are preferably substituted for one another, those within ±1 are more preferred, and those within ±0.5 are most preferred.

As outlined above, amino acid substitutions in an IL-19, IL-22 or IL-24 polypeptide can be based on the relative similarity of the amino acid side-chain substituents, for example, their hydrophobicity, hydrophilicity, charge, size, etc. Exemplary substitutions that take various of the foregoing characteristics into consideration in order to produce conservative amino acid changes resulting in silent changes within the present peptides, etc., can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral non-polar amino acids such as alanine, leucine, isoleucine, valine, proline, phenylalanine, tryptophan, and methionine.

IL-19, IL-22 or IL-24 variants having biological activities, in vivo or in vitro, that are similar or identical to those described herein, for example, the ability to induce or enhance differentiation and/or proliferation of erythroid and/or megakaryocyte progenitor cells are also useful in the methods of the present invention and as such are contemplated by the present invention. IL-19, IL-22 or IL-24 variants, while being functionally related, by definition include amino acid sequences that differ in one or more positions from the sequence as shown in SEQ ID NOs: 2, 4 and 6 respectively (with or without the signal sequence). Variants that are useful in the methods of the present invention can be generated by deletion, insertion, inversion, and/or substitution of one or more amino acid residues in said IL-19, IL-22 or IL-24 polypeptide. Such variants can generally be made by solid phase or recombinant techniques in which, for example, single or multiple conservative amino acid substitutions are made, according to Table 1. Generally, in the case of multiple substitutions, it is preferred that between 95% to 100% of the residues of an IL-19, IL-22 or IL-24 variant are identical to the corresponding contiguous sequence as shown in SEQ ID NOs: 2, 4, or 6 respectively (with or without the signal sequence); it is more preferable that between 96% to 100% of the residues of an IL-19, IL-22 or IL-24 variant are identical to the corresponding contiguous sequence as shown in SEQ ID NO: 2, 4, or 6 respectively (with or without the signal sequence); most preferably between 98% to 100% of the residues of a IL-19, IL-22, or IL-24 variant are identical to the corresponding contiguous sequence as shown in SEQ ID NO: 2, 4 or 6 respectively (with or without the signal sequence).

Another class of variant that may be useful in the methods of the present invention includes IL-19, IL-22 or IL-24 polypeptides as defined herein further comprising at least one oligopeptide or amino acid added onto the N-terminus and/or C-terminus. An “oligopeptide” is a chain of from 2 to about 250 amino acids connected at their N- and/or C-termini by peptide bonds. Suitable oligopeptides and amino acids are those that do not significantly decrease the biological activity of the polypeptide as defined herein and do not substantially detract from the desired pharmaceutical and pharmacological properties of the polypeptide. A preferred example of such a modification includes an IL-19, IL-22 or IL-24 polypeptide as defined herein further (SEQ ID NOs: 2, 4, or 6 respectively) comprising a leader sequences found in other polypeptides, such as pretrypsinogen leader sequence.

The IL-19, IL-22, or IL-24 polypeptides as defined herein can also be expressed and used in a modified form, such as a fusion protein or a “tagged” protein. IL-19, IL-22, or IL-24 fusion proteins represent a hybrid protein molecule not found in nature comprising a translational fusion or enzymatic fusion in which two or more different proteins, fragments, or variants thereof are covalently linked on a single polypeptide chain. Human serum albumin, the C-terminal domain of thrombopoietin, the C-terminal extension peptide of hCG, and/or a Fc fragment are examples of proteins which could be fused with IL-19, IL-22, or IL-24 polypeptides or variants thereof for use in the present invention. As used herein, “Fc fragment” of an antibody has the meaning commonly given to the term in the field of immunology. Specifically, this term refers to an antibody fragment which binds complement and is obtained by removing the two antigen binding regions (the Fab Fragments) from the antibody. Thus, the Fc fragment is formed from approximately equal sized fragments from both heavy chains, which associate through non-covalent interactions and disulfide bonds. The Fc Fragment includes the hinge regions and extends through the C_{H}2 and C_{H}3 domains to the C-terminus of the antibody.

In a preferred process for protein expression and subsequent purification, the IL-19, IL-22, or IL-24 gene (sequence as shown in SEQ ID NOs: 1, 3, 5 respectively) can be modified at the 5′ end to encode several histidine residues at the amino terminus of the protein resulting from its expression. This “histidine tag” enables a single-step protein purification method referred to as “immobilized metal ion affinity chromatography” (IMAC), essentially as described in U.S. Pat. No. 4,569,794. The IMAC method enables rapid isolation of substantially pure recombinant protein starting from a crude extract of cells that express a modified recombinant protein, as described above.

Synthesis

Functional fragments of IL-19, IL-22, or IL-24 polypeptides and variants thereof may be generated by any number of suitable techniques, including chemical synthesis of any portion of SEQ ID NOs: 2, 4 or 6, proteolytic digestion of IL-19, IL-22 or IL-24 polypeptides, propolypeptides or variants thereof, or most preferably, by recombinant DNA mutagenesis techniques well known to the skilled artisan. For example, in a preferred method, a nested set of deletion mutations are introduced into a nucleic acid sequence encoding a IL-19, IL-22 or IL-24 polypeptide such that varying amounts of the protein coding region are deleted, either from the amino terminal end or from the carboxyl end of the protein molecule. This method can also be used to create internal fragments of the intact protein in which both the carboxyl and amino terminal ends are removed. Several appropriate nucleases can be used to create such deletions, for example Bal 31 or mung bean nuclease. If desired, the resulting gene deletion fragments can be subcloned into any suitable vector for propagation and expression in any suitable host cell, bacterial, yeast, insect or mammalian.

Functional fragments of the proteins or full-length proteins disclosed herein may be produced as described above or using techniques well known in the art. Such proteins may be tested for biological activity using any suitable assay, for example, the ability to induce and/or enhance differentiation and/or proliferation of erythroid progenitor cells in vivo or in vitro.

Those skilled in the art will recognize that the gene encoding IL-19, IL-22 or IL-24 could be obtained by a plurality of recombinant DNA techniques including, for example, hybridization, polymerase chain reaction (PCR) amplification, or de novo DNA synthesis (4). Methods for constructing cDNA libraries in a suitable vector such as a plasmid or phage for propagation in prokaryotic or eukaryotic cells are well known to those skilled in the art. (4). Suitable cloning vectors are well known and are widely available.

The IL-19, IL-22 or IL-24 gene, or any fragment thereof, can be isolated from a tissue in which said gene is expressed, for example, placenta. In one method, mRNA is isolated, and first strand cDNA synthesis is carried out. A second round of DNA synthesis can be carried out for the production of the second strand. If desired, the double-stranded cDNA can be cloned into any suitable vector, for example, a plasmid, thereby forming a cDNA library. Oligonucleotide primers targeted to any suitable region of SEQ ID NO: 1, 3, or 5 for IL-19, IL-22 or IL-24 respectively can be used for PCR amplification. The PCR amplification comprises template DNA, suitable enzymes, primers, and buffers, and is conveniently carried out in a DNA Thermal Cycler (Perkin Elmer Cetus, Norwalk, Conn.). A positive result is determined by detecting an appropriately-sized DNA fragment following agarose gel electrophoresis. The proteins used in the present invention can be synthesized by a number of different methods, such as chemical methods well known in the art, including solid phase peptide synthesis or recombinant methods.

The proteins useful in the present invention can be produced by numerous means including recombinant DNA methods using the cloned IL-19, IL-22 or IL-24 gene. Recombinant methods are preferred if a high yield is desired. Expression of the cloned gene can be carried out in a variety of suitable host cells, well known to those skilled in the art. For this purpose, the IL-19, IL-22 or IL-24 gene is introduced into a host cell by any suitable means. While chromosomal integration of the cloned gene is within the scope of the present invention, it is preferred that the gene be cloned into a suitable extra-chromosomally maintained expression vector so that the coding region of the gene is operably-linked to a constitutive or inducible promoter.

The basic steps in the recombinant production of IL-19, IL-22 or IL-24 protein or variant thereof are:
a) constructing a natural, synthetic or semi-synthetic DNA encoding said protein or variant thereof;
b) integrating the DNA into an expression vector in a manner suitable for expressing the protein or variant thereof, either alone or as a fusion protein;
c) transforming or otherwise introducing said vector into an appropriate eukaryotic or prokaryotic host cell forming a recombinant host cell,
d) culturing said recombinant host cell in a manner to express the IL-19, IL-22 or IL-24 protein or or variant thereof; and
e) recovering and substantially purifying the IL-19, IL-22 or IL-24 protein or variant thereof by any suitable means, well known to those skilled in the art.

Vectors suitable to carry the nucleic acids of the present invention comprise RNA viruses, DNA viruses, lytic bacteriophages, lysogenic bacteriophages, stable bacteriophages, plasmids, viroids, and the like. The most preferred vectors are plasmids.

When preparing an expression vector the skilled artisan understands that the amount of nucleic acid or protein to be produced dictates, in part, the selection of the expression system. Regarding promoter sequences, inducible promoters are preferred because they enable high level, regulatable expression of an operably-linked gene. Other relevant considerations regarding an expression vector include whether to include sequences for directing the localization of a recombinant protein. For example, a sequence encoding a signal peptide preceding the coding region of a gene is useful for directing the extra-cellular export of a resulting polypeptide.

As previously mentioned, the IL-19, IL-22 or IL-24 polypeptides used in the methods of the present invention may be synthesized either by direct expression or as a fusion protein comprising the protein of interest as a translational fusion with another protein or peptide which may be removable by enzymatic or chemical cleavage. It is often observed in the production of certain peptides in recombinant systems that expression as a fusion protein prolongs the life span, increases the yield of the desired peptide, or provides a convenient means of purifying the protein. This is particularly relevant when expressing mammalian proteins in prokaryotic hosts. A variety of peptidases (e.g. enterokinase and thrombin) which cleave a polypeptide at specific sites or digest the peptides from the amino- or carboxy-termini (e.g. diaminopeptidase) of the peptide chain are known. Furthermore, particular chemicals (e.g. cyanogen bromide) will cleave a polypeptide chain at specific sites.

Exemplary mammalian host cells suitable for use in the present invention include, but are not limited to, HepG-2 (ATCC HB 8065), CV-1 (ATCC CCL 70), LC-MK₂ (ATCC CCL 7.1), 3T3 (ATCC CCL 92), CHO-K1 (ATCC CCL 61), HeLa (ATCC CCL 2), RPMI8226 (ATCC CCL 155), H4IIEC3 (ATCC CCL 1600), C127I (ATCC CCL 1616), HS-Sultan (ATCC CCL 1484), and BHK-21 (ATCC CCL 10). Transfection of mammalian cells with vectors can be performed by a plurality of well-known processes including, but not limited to, protoplast fusion, calcium phosphate co-precipitation, electroporation and the like (4). The recombinantly-produced protein may be purified from cellular extracts of transformed cells by any suitable means.

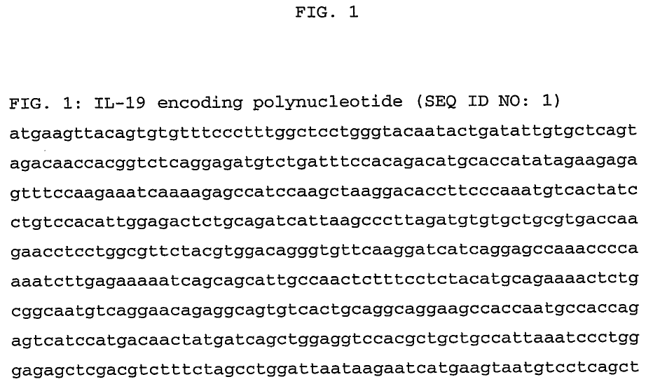

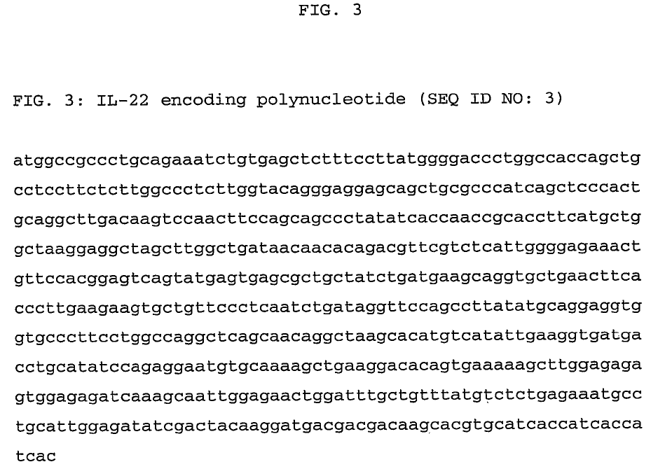

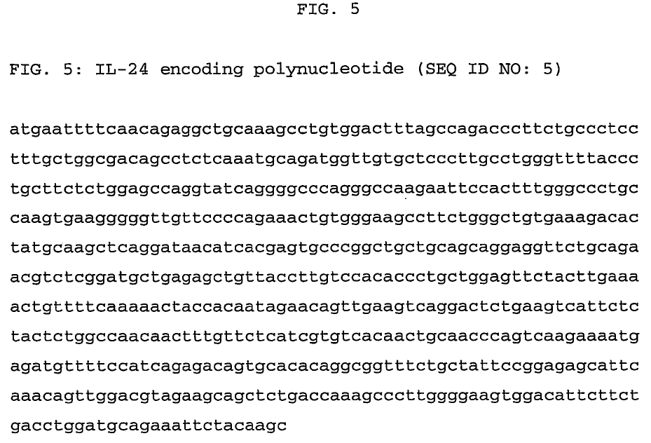

The IL-19, IL-22 and IL-24 cDNA (sequence shown in SEQ ID NO: 1, 3 and 5 respectively shown in FIGS. 1, 3 and 5) and related nucleic acid molecules that encode SEQ ID NO: 2, 4, or 6, or variants thereof (with or without the signal peptide sequence), may be produced by chemical synthetic methods. Fragments of the DNA sequence corresponding to the gene of interest may be generated using a conventional DNA synthesizing apparatus, such as the Applied Biosystems Model 380A or 380B DNA synthesizers (Applied Biosystems, Inc. Foster City, Calif.) using phosphoramidite chemistry, thereafter ligating the fragments so as to reconstitute the entire gene. Alternatively, phosphotriester chemistry may be employed to synthesize the nucleic acids of this invention (7).

In an alternative methodology, namely PCR, the DNA sequences disclosed and described herein, comprising, for example, a portion or all of SEQ ID NO: 1, 3 or 5 can be produced from a plurality of starting materials. For example, starting with a cDNA preparation derived from a tissue that expresses the IL-19, IL-22 or IL-24 gene. Using PCR, any region of the IL-19, IL-22 or IL-24 gene can be targeted for amplification such that full or partial length gene sequences may be produced.

Signal Sequence

The polypeptides of the invention may have a signal peptide sequence to enable protein transport within the cell (FIGS. 2, 4 and 6); however, this signal peptide sequence is not present in the mature polypeptides as they exist when transported outside the cell. Alternatively, the polypeptide of the invention may be made without a signal peptide sequence. Either way, the mature polypeptide does not possess the signal peptide sequence.

A signal peptide, comprised of about 10-30 hydrophobic amino acids, targets the nascent protein from the ribosome to the endoplasmic reticulum (ER). Once localized to the ER, the proteins can be further directed to the Golgi apparatus within the cell. The Golgi distributes proteins to vesicles, lysosomes, the cell membrane, and other organelles. Proteins targeted to the ER by a signal sequence can be released from the cell into the extracellular space. Vesicles containing proteins to be moved outside the cell can fuse with the cell membrane and release their contents into the extracellular space via a process called exocytosis. Exocytosis can occur constitutively or after receipt of a triggering signal. In the latter case, the proteins are stored in secretory vesicles until exocytosis is triggered. Proteins that transit through this pathway are either released into the extracellular space or retained in the plasma membrane.

The common structure of signal peptides from various proteins is typically described as a positively charged n-region, followed by a hydrophobic h-region and a neutral but polar c-region. The (−3, −1) rule states that the residues at positions −3 and −1 (relative to the signal peptide cleavage site) must be small and neutral for cleavage to occur correctly.

In many instances the amino acids comprising the signal peptide are cleaved off the protein during transport or once its final destination has been reached. Specialized enzymes, signal peptidases, are responsible for the removal of the signal peptide sequences from proteins. These enzymes are activated once the signal peptide has directed the protein to the desired location.

Polypeptides of the present invention may be produced recombinantly. In general, the signal sequence may be a component of an expression vector, or it may be a part of the DNA encoding the polypeptide of the invention that is inserted into such a vector. For E. coli expression, the signal sequence may be a prokaryotic signal sequence selected, for example, from the group of the alkaline phosphatase, penicillinase, Ipp, or heat-stable enterotoxin II leaders. For yeast secretion the signal sequence may be, e.g., the yeast invertase leader, alpha factor leader (including Saccharomyces and Kluyveromyces cc-factor leaders, the latter described in U.S. Pat. No. 5,010,182), or acid phosphatase leader, the C. albicans glucoamylase leader (EP 362,179), or the signal described in WO 90/13646. In mammalian cell expression, mammalian signal sequences may be used to direct secretion of the protein, such as signal sequences from secreted polypeptides of the same or related species as well as viral secretory leaders.

The usefulness of a IL-19, IL-22 or IL-24 agonist, IL-19, IL-22 or IL-24 antagonist, IL-19, IL-22 or IL-24 polypeptide or variant thereof, or a composition comprising, or alternatively consisting of or essentially consisting of, at least one IL-19, IL-22 or IL-24 agonist, IL-19, IL-22 or IL-24 antagonist, isolated and purified IL-19, IL-22 or IL-24 polypeptide or variant thereof for the purpose of modifying a hematopoietic disorder in a mammal, human, or patient in need of such treatment can be determined by one skilled in the art without undue experimentation by application of the methods or assays described herein or otherwise known in the art. Similarly, the usefulness of an IL-19, IL-22 or IL-24 agonist, IL-19, IL-22 or IL-24 antagonist, IL-19, IL-22 or IL-24 polypeptide or variant thereof, or a composition comprising, or alternatively consisting of or essentially consisting of, at least one IL-19, IL-22 or IL-24 agonist, IL-19, IL-22 or IL-24 antagonist, IL-19, IL-22 or IL-24 polypeptide or variant thereof, of the present invention in the methods of the present invention can be assessed or quantified using the in vitro models or in vivo models of hematopoiesis as described herein (see Examples) or assays otherwise known in the art.

Cell-based systems can be used to identify compounds that may act to ameliorate hematopoietic disorder symptoms. Such cell systems can include, for example, recombinant or non-recombinant cell, such as cell lines, that express the IL-19, IL-22 or IL-24 gene. In utilizing such cell systems, cells that express IL-19, IL-22 or IL-24 may be exposed to a compound suspected of exhibiting an ability to ameliorate hematopoietic disorder symptoms, at a sufficient concentration and for a sufficient time to elicit such an amelioration of such symptoms in the exposed cells.

After exposure the cells can be assayed to measure alterations in the expression of the IL-19, IL-22 or IL-24 gene, e.g., by assaying cell lysates for IL-19, IL-22 or IL-24 mRNA transcripts or for IL-19, IL-22 or IL-24 gene products expressed by the cell.

In addition, animal-based systems or models for a mammalian hematopoietic disorder, for example, transgenic mice containing a human or altered form of IL-19, IL-22 or IL-24 gene, may be used to identify capable of ameliorating symptoms of the disorder. Such animal models may be used as test substrates for the identification of drugs, pharmaceuticals, therapies and interventions. For example, animal models may be exposed to a compound suspected of exhibiting an ability to ameliorate symptoms, at a sufficient concentration and for a sufficient time to elicit such an amelioration of the symptoms of the hematopoietic disorder. An animal's response to a particular treatment may be monitored by assessing reductions in the symptoms attributable to the disorder. Treatments that favorably affect hematopoietic disorder-like symptoms may be considered as candidates for human therapeutic intervention in such a disorder. Dosages of test agents may be determined by deriving dose-response curves

In one embodiment, methods of the present invention comprise contacting a compound to a cell, measuring the level of IL-19, IL-22 or IL-24 gene expression, gene product expression, or gene product activity, and comparing the level to the level of IL-19, IL-22 or IL-24 gene expression, gene product expression, or gene product activity produced by the cell in the absence of the compound. If the level obtained in the presence of the compound differs from that obtained in its absence, a compound that modulates the expression of the mammalian IL-19, IL-22 or IL-24 gene and/or the synthesis or activity of mammalian IL-19, IL-22 or IL-24 gene products has been identified.

In an alternative embodiment, methods of the present invention comprise administering a compound to a host, and measuring the level of IL-19, IL-22 or IL-24 gene expression, gene product expression, or gene product activity. The measured level is compared to the level of IL-19, IL-22 or IL-24 gene expression, gene product expression, or gene product activity in a host that is not exposed to the compound. If the level obtained when the host is exposed to the compound differs from that obtained when the host is not exposed to the compound, a compound that modulates either the expression of the mammalian IL-19, IL-22 or IL-24 gene, the synthesis or activity of the IL-19, IL-22 or IL-24 gene product, the compound may be used in the methods of the present invention.

Methods of the present invention can comprise, for example, administering compounds which modulate the expression of a mammalian IL-19, IL-22 or IL-24 gene and/or the synthesis and/or the activity of a mammalian IL-19, IL-22 or IL-24 gene product, so that symptoms of a hematopoietic disorder are ameliorated. Alternatively, in those instances whereby the mammalian hematopoietic disorder results from IL-19, IL-22 or IL-24 gene mutations, such methods can comprise supplying the mammal with a nucleic acid molecule encoding an unimpaired IL-19, IL-22 or IL-24 gene product such that an unimpaired IL-19, IL-22 or IL-24 gene product is expressed and symptoms of the disorder are ameliorated.

For therapeutic utility, an effective amount of at least one IL-19, IL-22 or IL-24 agonist, IL-19, IL-22 or IL-24 antagonist, isolated and purified IL-19, IL-22 or IL-24 polypeptide or variant thereof, or a composition comprising, or alternatively consisting of or consisting essentially of, an effective amount of at least one IL-19, IL-22 or IL-24 agonist, IL-19, IL-22 or IL-24 antagonist, IL-19, IL-22 or IL-24 polypeptide or variant thereof is administered to a mammal, human or patient in need thereof in a dose between about 0.1 and 1000 μg/kg. In practicing the methods contemplated by this invention, the IL-19, IL-22 or IL-24 agonists, IL-19, IL-22 or IL-24 antagonists, IL-19, IL-22 or IL-24 polypeptides or variants thereof, or compositions comprising, or alternatively consisting of or consisting essentially of at least one IL-19, IL-22 or IL-24 agonist, IL-19, IL-22 or IL-24 antagonist, IL-19, IL-22 or IL-24 polypeptide or variant thereof as defined herein can be administered in multiple doses per day, in single daily doses, in weekly doses, or at any other regular interval. The amount per administration and frequency of administration will be determined by a physician and depend on such factors as the nature and severity of the disease, and the age and general health of the patient.

The present invention also provides a pharmaceutical IL-19, IL-22, or IL-24 composition comprising as the active agent an IL-19, IL-22 or IL-24 agonist, IL 19, IL-22 or IL-24 antagonist, IL-19, IL-22 or IL-24 polypeptide or variant thereof and a pharmaceutically acceptable solid or liquid carrier. For example, at least one IL-19, IL-22 or IL-24 agonist, IL-19, IL-22 or IL-24 antagonist, IL-19, IL-22 or IL-24 polypeptide or variant thereof can be admixed with conventional pharmaceutical carriers and excipients, and used in the form of tablets, capsules, elixirs, suspensions, syrups, wafers, parenteral formulations, and the like. The compositions will contain from about 0.1% to 90% by weight of at least one active IL-19, IL-22 or IL-24 agonist, IL-19, IL-22 or IL-24 antagonist, IL-19, IL-22 or IL-24 polypeptide or variant thereof and more generally from about 10% to 30%. The compositions may contain common carriers and excipients such as corn-starch or gelatin, lactose, sucrose, microcrystalline cellulose, kaolin, mannitol, dicalcium phosphate, sodium chloride, and alginic acid.

As a general proposition, the total pharmaceutically effective amount of at least one IL-19, IL-22 or IL-24 agonist, IL-19, IL-22 or IL-24 antagonist, IL-19, IL-22 or IL-24 polypeptide or variant thereof administered parenterally to a patient per dose will be in the range of about 1 μg/kg/day to 10 mg/kg/day, particularly 2 mg/kg/day to 8 mg/kg/day, more particularly 2 mg/kg/day to 4 mg/kg/day, even more particularly 2.2 mg/kg/day to 3.3 mg/kg/day, and finally 2.5 mg/kg/day, although, as noted above, this will be subject to therapeutic discretion. More preferably, this dose is at least 0.01 mg/kg/day. If given continuously a IL-19, IL-22 or IL-24 agonist, IL-19, IL-22 or IL-24 antagonist, IL-19, IL-22 or IL-24 polypeptide or variant thereof is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions comprising at least one IL-19, IL-22 or IL-24 agonist, IL-19, IL-22 or IL-24 antagonist, IL-19, IL-22 or IL-24 polypeptide or variant thereof may be administered orally, rectally, intracranially, parenterally, intracistemally, intravaginally, intraperitoneally, topically (as by powders, ointments, drops or transdermal patch), transdermally, intrathecally, bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein includes, but is not limited to, modes of administration which include intravenous, intramuscular, intraperitoneal, intrasternal, subcutaneous and intra-articular injection, infusion and implants comprising (alternatively consisting of or consisting essentially of) at least one IL-19, IL-22 or IL-24 agonist, IL-19, IL-22 or IL-24 antagonist, IL-19, IL-22 or IL-24 polypeptide or variant thereof.

The compounds can be formulated for oral or parenteral administration. A preferred parenteral formulation for subcutaneous administration would comprise a buffer (e.g., phosphate, citrate, acetate, borate, TRIS), salt (e.g., NaCl, KCl), divalent metal (e.g., Zn, Ca), and isotonicty agent (e.g., glycerol, mannitol), detergent (e.g., polyoxyethylene sorbitan fatyy acid esters, poloxamer, ddicusate sodium, sodium lauryl sulfate), antioxidants (e.g., ascorbic acid), and antimicrobial agent (e.g., phenol, m-cresol, alcohol, benzyl alcohol, butylparben, methylparaben, ethylparaben, chlorocresol, phenoxyethanol,phenylethyl alcohol, propylparaben).

For intravenous (IV) use, at least one IL-19, IL-22 or IL-24 agonist, IL-19, IL-22 or IL-24 antagonist, IL-19, IL-22 or IL-24 polypeptide or variant thereof is administered in commonly used intravenous fluid(s) and administered by infusion. Such fluids, for example, physiological saline, Ringer's solution or 5% dextrose solution can be used.

For intramuscular preparations, a sterile formulation, preferably a suitable soluble salt form of at least one IL-19, IL-22 or IL-24 agonist, IL-19, IL-22 or IL-24 antagonist, IL-19, IL-22 or IL-24 polypeptide or variant thereof such as the hydrochloride salt, can be dissolved and administered in a pharmaceutical diluent such as pyrogen-free water (distilled), physiological saline, or a 5% glucose solution. A suitable insoluble form of the compound may be prepared and administered as a suspension in an aqueous base or a pharmaceutically acceptable oil base, e.g. an ester of a long chain fatty acid such as ethyl oleate.

An IL-19, IL-22 or IL-24 agonist, IL-19, IL-22 or IL-24 antagonist, IL-19, IL-22 or IL-24 polypeptide or variant thereof is also suitably administered by sustained-release systems. Suitable examples of sustained-release compositions include semi-permeable polymer matrices in the form of shaped articles, e.g., films, or microcapsules. Sustained-release matrices include, but are not limited to, polylactides (U.S. Pat. No. 3,773,919, EP 58,481). Other sustained-release compositions also include liposomally entrapped modified IL-19, IL-22 or IL-24 polypeptides or variants thereof. Such liposomes are prepared by methods known for example: U.S. Pat. Nos. 4,485,045 and 4,544,545; and EP 102,324. Ordinarily, the liposomes are of the small (about 200-800 Angstroms) unilamellar type in which the lipid content is greater than about 30 mol percent cholesterol, the selected proportion being adjusted for the optimal therapy.

For parenteral administration, in one embodiment, the IL-19, IL-22 or IL-24 agonist, IL-19, IL-22 or IL-24 antagonist, IL-19, IL-22 or IL-24 polypeptide or variant thereof may be formulated generally by mixing it at the desired degree of purity, in a unit dosage injectable form (solution, suspension, or emulsion), with a pharmaceutically acceptable carrier (i.e., one that is non-toxic to recipients at the dosages and concentrations employed and is compatible with other ingredients of the formulation). Preferably, the formulation does not include oxidizing agents and other compounds that are known to be deleterious to polypeptides.

Generally, the formulations are prepared by contacting at least one IL-19, IL-22 or IL-24 agonist, IL-19, IL-22 or IL-24 antagonist, IL-19, IL-22 or IL-24 polypeptide or variant thereof uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers for example phosphate, citrate, succinate, acetic acid, and other organic acids or their salts; antioxidants such as ascorbic acid; low molecular weight (less than about ten residues) polypeptides, e.g., polyarginine or tripeptides; proteins, for example serum albumin, gelatin, or immunoglobulins; hydrophilic polymers such as polyvinylpyrrolidone; amino acids, for example glycine, glutamic acid, aspartic acid, or arginine; monosaccharides, disaccharides, and other carbohydrates including cellulose or its derivatives, glucose, manose, or dextrins; chelating agents such as EDTA; sugar alcohols for example mannitol or sorbitol; counterions such as sodium; and/or nonionic surfactants for example polysorbates, poloxamers, or PEG.

At least one IL-19, IL-22 or IL-24 agonist, IL-19, IL-22 or IL-24 antagonist, IL-19, IL-22 or IL-24 polypeptide or variant thereof is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of salts of the particular active ingredient(s).

Compositions to be used for therapeutic administration must be sterile. Sterility is readily accomplished by filtration through sterile filtration membranes (e.g., 0.2 micron membranes). Therapeutic compositions generally are placed into a container having a sterile access port, for example, an intravenous solution bag or vial having a stopper pierceable by a hypodermic injection needle.

Pharmaceutically useful IL-19, IL-22 or IL-24 compositions ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous solution of one of an IL-19, IL-22 or IL-24 composition, and the resulting mixture is lyophilized. The infusion solution is prepared by reconstituting the lyophilized polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In addition the therapeutic methods of the present invention may also be employed, alone or in combination with other cytokines, soluble Mpl receptor, hematopoietic factors, interleukins, growth factors, chimeras thereof (e.g., myelopoietin) or antibodies thereto or in combination with any of the soluble receptors or augmentors thereof in the treatment of hematopoietic disease states. It is anticipated that the inventive therapeutic methods will prove useful in treating some forms of thrombocytopenia, anemia, leukemia, bone marrow transplant, and in combination with general stimulators of hematopoiesis, such as IL-3 or GM-CSF. Other megakaryocytic stimulatory factors, i.e., meg-CSF, stem cell factor (SCF), leukemia inhibitory factor (LIF), oncostatin M (OSM), or other molecules with megakaryocyte stimulating activity may also be employed with at least one IL-19, IL-22 or IL-24 agonist, IL-19, IL-22 or IL-24 antagonist, IL-19, IL-22 or IL-24 polypeptide or variant thereof. Additional exemplary cytokines or hematopoietic factors for such co-administration include: IL-1 alpha, IL-1 beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-20, colony stimulating factor-1 (CSF-1), M-CSF, SCF, GM-CSF, granulocyte colony stimulating factor (G-CSF), EPO, interferon-alpha (IFN-alpha), consensus interferon, IFN-beta, IFN-gamma, thrombopoietin (TPO), angiopoietins, e.g., Ang-1, Ang-2, Ang-4, Ang-Y, the human angiopoietin-like polypeptide, vascular endothelial growth factor (VEGF), angiogenin, bone morphogenic protein-1, 2, 3, 4, 5, 6, 7, 8, 9, 10, 11, 12, 13, 14 or 15, bone morphogenic protein receptor 1a, bone morphogenic protein receptor 1b, brain derived neurotrophic factor, ciliary neutrophic factor, ciliary neutrophic factor receptor, epidermal growth factor, epithelial-derived neutrophil attractant, fibroblast growth factor 4, 5, 6, 7, 8, 8a, 8b, 8c, 9 or 10, fibroblast growth factor acidic, fibroblast growth factor basic, glial cell line-derived neutrophic factor receptors, heparin binding epidermal growth factor, hepatocyte growth factors, hepatocyte growth factor receptors, insulin-like growth factor 1, insulin-like growth factor receptors, insulin-like growth factor 11, insulin-like growth factor binding protein, keratinocyte growth factor, leukemia inhibitory factor, leukemia inhibitory factor receptor a, nerve growth factor, nerve growth factor receptors, neurotrophin-3, neurotrophin-4, placenta growth factor, placenta growth factor 2, platelet-derived endothelial cell growth factor, platelet derived growth factors including, but not limited to, platelet derived growth factor A chain, platelet derived growth factor AA, platelet derived growth factor AB, platelet derived growth factor B chain, platelet derived growth factor BB, and platelet derived growth factor receptors, pre-B cell growth stimulating factor, stem cell factor receptor, tumor necrosis factors, including TNFO, TNFI, TNF2, transforming growth factor u, transforming growth factor P, transforming growth factor P1, P1.2, P2, P3 or P5, latent transforming growth factor P1, transforming growth factor P binding protein 1, transforming growth factor P binding protein 2, transforming growth factor P binding protein 3, tumor necrosis factor receptor type 1, tumor necrosis factor receptor type 2, urokinase-type plasminogen activator receptor, vascular endothelial growth factor, and chimeric proteins and biologically or immunologically active fragments thereof. It may further be useful to administer, either simultaneously or sequentially, an effective amount of a soluble mammalian Mpl receptor, which appears to have an effect of causing megakaryocytes to fragment into platelets once the megakaryocytes have reached mature form. Thus, administration of an IL-19, IL-22 or IL-24 composition in combination with at least one of the additional factors provided hereinabove in combination with administration of the soluble Mpl receptor (to inactivate the ligand and allow the mature megakaryocytes to produce platelets) is expected to be a particularly effective means of stimulating platelet production. The dosage recited above would be adjusted to compensate for such additional components in the therapeutic composition. Administration in combination with one or more further therapeutic agents includes simultaneous (concurrent) and consecutive administration in any order. Progress of the treated patient can be monitored by assays provided herein or otherwise known in the art.

The following examples more fully describe the present invention. Those skilled in the art will recognize that the particular reagents, equipment, and procedures described are merely illustrative and are not intended to limit the present invention in any manner.

### EXAMPLES

### Example 1

### Transgenic Rodent Development

A. Transgene construction.

Polymerase chain reaction (PCR) primers are synthesized according to standard methods and used to separately amplify the IL-24 coding region and the IL-19 coding region from plasmids containing the full length coding region plus surrounding sequences or from genomic DNA:

For IL-24 an exemplary set of PCR primers are:

| | |
|---|---|
| 5′ GAGACTGAGAGATGAATTTTC 3′ | (SEQ ID NO. 7) |
| 5′ GACATTCAGAGCTTGTAGAATTTC 3′ | (SEQ ID NO. 8) |

For IL-19 an exemplary set of PCR primers are:

| | |
|---|---|
| 5′ CAAGTGAGAGGCATGAAGTTAC 3′ | (SEQ ID NO. 9) |
| 5′ CCTTGTCATCAAGCTGAGGAC 3′ | (SEQ ID NO. 10) |

For IL-22 an exemplary set of PCR primers may be found in Reference 3. Many alternative PCR primer sets may be designed by one skilled in the art. The primers may incorporate restriction enzyme sites to accommodate cloning into a plasmid.

B. Transgenic Animal Development.

Transgenic mice were generated using established techniques [Hogan, B. et al. (1986) Manipulating the Mouse Embryo: A Laboratory Manual. Cold Spring Harbor Laboratory, N.Y.] as modified by Ref. 8. Briefly, a 6.4 kb DNA fragment encompassing the human apolipoprotein E (hApoE) gene promoter-5′ hApoE untranslated region-IL-19, IL-22 OR IL-24/FLAG-hepatic control region (HCR) fusion gene is excised from plasmid pLIV7-IL-19, IL-22 OR IL-24 by digestion with Sal I and Spe I and purified by gel electrophoresis and glass bead extraction. The purified DNA fragment encompassing the hApoE gene promoter-5′ hApoE untranslated region-(IL-19, IL-22 or IL-24)-HCR fusion gene is microinjected into the male pronuclei of newly fertilized one-cell-stage embryos (zygotes) of the FVB/N strain. The embryos are cultured in vitro overnight to allow development to the two-cell-stage. Two-cell embryos are then transplanted into the oviducts of pseudopregnant ICR strain mice to allow development to term. To test for the presence of the transgene in the newborn mice, a small piece of toe is removed from each animal and digested with proteinase K to release the nucleic acids. A sample of the toe extract is subsequently subjected to PCR analysis using primers specific for the hApoE untranslated region to identify transgene containing mice. Five founder transgenic mice are identified for each gene. Each of these founders is bred to produce F1 and F2 progeny.

### Example 2

### In Vitro Testing for Hematopietic Modulators

A. Human Megakaryocyte Assay

Test polypeptides (e.g., IL-19, IL-22 and IL-24 polypeptides) can be assayed for their ability to stimulate development of human megakaryocytes from CD34+ progenitor cells. CD34+ selected cells are obtained from bone marrow (9) and are incubated in Iscove's modified Dulbecco's medium (IMDM; GIBCO) with 2 mM Glutamine, 2-mercaptoethanol (10⁻⁴ M), 1% bovine serum albumin, low density lipoprotein (40 μg/ml, Sigma); bovine pancreatic insulin (10 μg/ml), human transferrin (200 μg/ml), human recombinant thrombopoietin (50 ng/ml, R&D Systems); human recombinant stem cell factor (50 ng/ml, R&D Systems), human recombinant IL-3 (10 ng/ml, R&D System) and 1.1 mg/ml collagen. CD34+ cells are plated at 3300-cells/ml final concentrations on 2 well chamber slides. Cells are incubated at 37° C. for 12 days in humidified boxes in 5% CO₂ in air, fixed directly to the culture wells with 1:3 methanol:acetone solution, and incubated with a monoclonal antibody, anti-GPIIb/IIIa, (StemCell Technologies, Vancouver, Canada). The immune reaction was developed with biotin-conjugated goat anti-mouse IgG followed by avidin-alkaline phosphatase conjugate, identified by pink color, are counted with an inverted phase microscope at 100× magnification. Results are presented as the average number of megakaryocytes per well +/− standard error of the mean (SEM).

B. Proliferation/Differentiation Activity of IL-19, IL-22 and IL-24 Polypeptides on Human Hematopoietic Progenitors

Human bone marrow CD34+ cells from Poietic, BioWhittaker are incubated in Iscove's modified Dulbecco's medium (IMDM; GIBCO) supplemented with 0.9% Methylcellulose, 2 mM Glutamine, 2-mercapto-ethanol (10⁻⁴ M), 1% bovine serum albumin, bovine pancreatic insulin (10 □g/ml), human transferrin (200 □g/ml) and various concentration of human stem cell factor, IL-3, EPO, and GM-CSF. CD34+ cells are plated at 1000 cells/ml final concentration in 35-mm dishes. Cells are incubated at 37° C. for 14-16 days in 5% CO₂ in air. Colonies are scored under an inverted microscope. Treatment with test polypeptide(s) (e.g.,IL-22, IL-19, IL-24 polypeptide) (200 ng/ml) may result in colony size increase.

C. Assay for Liquid Bone Marrow Culture

CD34+ human bone marrow cells are purchased from Poietic, BioWhittaker and incubated in Iscove's modified Dulbecco's medium (IMDM; GIBCO) supplemented with 30% fetal bovine serum, antibiotics, 2 mM Glutamine, 2-mercapto-ethanol (10⁻⁴ M), and various concentrations of human stem cell factor, IL-3, EPO and/or GM-CSF. CD34+ cells are plated in U-bottomed 96 well plates at 5000 cells/well and cultured at 37° C., 5% CO₂ for 10 days with a breathable membrane to prevent evaporation. Feeding occurs at days 4 and 7 by replacing 80% of the medium with fresh medium. At day 10, the cells are transferred to V-bottomed plates and stained for CD41 (FITC) and CD36 (PE). Cells are then acquired on a flow cytometer in timed acquisition mode and compared to the negative controls. Treatment with test polypeptide (e.g., IL-19, IL-22, or IL-24 polypeptide) (200 ng/ml) may stimulate proliferation of CD34+ cells.

### Example 3

### In Vivo Testing for Hematopietic Modulators

A. Recovery of Blood Cells after Bone Marrow Transplantation.

Bone marrow is harvested by gentle flushing of the hind limbs of normal 8- to 10-week-old Balb-C mice (Harlan Sprague Dawley) using RPMI medium (GIBCO) containing 10% fetal calf serum. For some experiments, donor mice are pretreated with 5-fluorouracil (5-FU) at 150-mg/kg-body weight intraperitoneally 3 days before harvesting BM for infusion. After total body irradiation with 10.8 Gy (¹³⁷Cs at 126cGy/min, split dose with a minimum of 3 hours between doses), 1×10⁶ bone marrow cells are injected intravenously into sub-lethally irradiated mice. The polypeptide to be tested (250 μg/kg body weight) is diluted in PBS and injected subcutaneously in 0.2-ml volume daily starting on the same day as irradiation and infusion of donor bone marrow cells. Control mice receive the same volume of PBS. Mice are weighed every 2 to 4 days during the post-transplantation period. Hematologic analysis of leukocyte cell counts and platelet counts are performed on orbit bleeds on a CDC Mascot™ machine. Blood smears are stained with Wright-Giemsa using standard methods and examine at 100× for differentiation analysis. Peripheral blood hematocrits are performed by spinning capillary tubes for 5 minutes in a Model MB Micro-Capillary Centrifuge. Accordingly, test polypeptides can be used to accelerate recovery of peripheral blood cell counts.

B. Recovery of Blood Cells after Combined Chemo-/Radiation Therapy

Eight- to ten-week old Balb/C mice (Harlan Sprague Dawley) are administered 5-fluorouracil at 150-mg/kg body weights intraperitoneally 3 days before sub-lethal irradiation (0.6 Gy total body irradiation for 20-22 mg mouse). Test polypeptide(s) are injected sub-cutaneously in 0.2 ml volumes daily starting on the same day as irradiation. Negative control mice receive the same volume of PBS as the treated mice. Test polypeptide administration lasts for 14 days. The mice are analyzed at 7 days and 14 days post-radiation. Mice are weighed every 2 to 4 days during the post-radiation period. Hematologic analysis of leukocyte cell counts and platelet counts are performed on orbit bleeds on a CDC Mascot™ machine. Blood smears are stained with Wright-Giemsa using standard methods and examine at 100× for differentiation analysis. Peripheral blood hematocrits were performed by spinning capillary tubes for 5 minutes in a Model MB Micro-Capillary Centrifuge. Test polypeptides may be useful in accelerating recovery of peripheral blood cell counts after chemo-/radiation therapy

C. Treatment of Anemia

Various animal models of anemia and hematopoietic disorder are known in the art and generally accepted as being indicative of the anemic condition. For instance, the exhypoxic polycythemic mouse bioassay may be used to quantify the incorporation of 5′ Fe(iron) into newly synthesized red blood cells as a measure of the increase in erythropoiesis in mice in response to an exogenously administered test sample The assay, is described in WO 0024893 (assay herein incorporated by reference).

The test agent(s) may be administered by any of several routes of administration (e.g. i.v., s.c., i.p., or by minipump or cannula) and suitable test animals include normal mice as well as IL-19, IL-22 or IL-24 transgenic mice similar to those described in Example 1. Controls for non-specific effects for these treatments are done using vehicle with or without the active agent of similar composition in the same type animal monitoring the same parameters.

### Example 4

### Proliferation of IL-19, IL-22 or IL-24 Splenocytes in a Mixed Lymphocyte Reaction

In a mixed lymphocyte reaction assay, splenocytes from DBA/2 mice (Harlan Sprague Dawley) may be used as stimulator cells after being treated with mitomycin C. The responder cells are splenocytes isolated from C57BL/6 mice (Harlan Sprague Dawley) transplanted with the IL-19, IL-22 or IL-24 gene or naïve mice. The suspensions of responder T cells are cultured with allogeneic stimulator lymphocytes. The activating stimulus is the foreign histocompatibility antigen (usually MHC class I or class II molecules) expressed on the allogenic stimulator cells.

In brief, splenocytes from DBA/2 are added to 96-well plates at 1×10⁶ cells per well in RPMI+10% FBS and Pen/Strep. Splenocytes from either age matched C57BL/6 naïve mice or retroviral expressed IL-19, IL-22 or IL-24 mice are added as responder cells to wells at either 0.5, 1, 2, 4, or 8×10⁵ cells per well. Control wells contained DBA stimulator splenocytes alone or C57BL/6 responder spleenocytes alone. After 72 hours in vitro, wells are pulse labeled with 1 μCi of tritiated thymidine. After 18 hrs, cells can be harvested and counted.

### Example 5

### Proliferation And Cytokine Secretion of IL-19 IL-22 or IL-24 Expressing Splenocytes Upon Antigenic Stimulation

Flat bottom 96 well plates are coated with 100 μl media (RPMI, 10% FBS) containing 5 μg/ml α-CD3. Plates are coated for 1.5 hrs. at 37° C., aspirated, and washed 2× in PBS. Then, 4×10⁵ spleen cells in a 100 μl volume of media are added to each well and plates are incubated for 48 hrs. at 37° C. After plates are centrifuged at 1200 rpm for 5 min., 100 μl of supernatant from each well is removed and transferred to 96-well U-bottom plates of which 10 μl is used for the cytokine secretion immunoassay according to standard procedures. Remainder of cells are pulse-labeled with 1 μCi of 3H-thymidine/well and incubated for another 24 hrs prior to counting. In addition to activation of splenocytes by anti-CD3, other stimuli can be tested in the same manner. Preferred stimuli for testing include dilutions of 2.5 ng/ml IL-2, dilutions of 8 μg/ml ConA, dilutions of PMA with 1 μM ionomycin, and 100 μg/ml LPS.

### Example 6

### Exposure of Transgenic Mice to Sub-Lethal Doses of Radiation

Wild type and transgenic mice of both genders are irradiated at 600 cGy. The mice are analyzed at 3, 7, 10 days and 14 days post-radiation. Mice are weighed every 2 days during the post-radiation period. Hematologic analysis of leukocyte cell counts and platelet counts are performed on orbit bleeds on a CDC Mascot™ machine. Blood smears are stained with Wright-Giemsa using standard methods and examine at 100× for differentiation analysis. Peripheral blood hematocrits were performed by spinning capillary tubes for 5 minutes in a Model MB Micro-Capillary Centrifuge. IL-19, IL-22 or IL-24 may be used to accelerate the recovery of peripheral blood cell counts after exposure to sub-lethal doses of radiation.

### REFERENCES

1. Dumoutier, L., et al. J. Immunology. (2001). 167:3545-3549.
2. Kyte and Doolittle. 1982, J. Mol. Biol., 157:105-132.
3. Dumoutier, et al. Genes and Immunity (2000). 1:488-494.
4. Sambrook, et al. Molecular Cloning: A Laboratory Manual, 2d Ed. Chap. 14 (1989).
5. Smith and Waterman, J. Mol. Biol. (1985). 147:195.
6. Altschul et al. J. Mol. Biol. (1990). 215:403.
7. M. J. Gait, ed., Oligonucleotide Synthesis, A Practical Approach, (1984).
8. Fox and Solter, Mol. Cell. Biol. (1988). 8:5470.
9. Hokom, M. H., Choi, E., Nichol, J. L., Hornkohl, A., Arakawa and Hunt, Molecular Biology of Haematopoiesis (1994). 3:15.

1. A method of increasing the number of one or more type(s) of hematopoietic progenitor cells in a mammal in need thereof comprising administering a therapeutically effective amount of a polypeptide comprising an amino acid sequence selected from the group consisting of: SEQ ID NO: 2, SEQ ID NO: 4, SEQ ID NO: 6, amino acids about 25-177 of SEQ ID NO: 2, amino acids about 28-179 of SEQ ID NO: 4, and amino acids about 26-206 of SEQ ID NO: 6.

2. A method of increasing the number of one or more types of mature hematopoietic cells in a mammal in need thereof comprising administering a therapeutically effective amount of a polypeptide comprising an amino acid sequence selected from the group consisting of: SEQ ID NO: 2, SEQ ID NO: 4, SEQ ID NO: 6, amino acids about 25-177 of SEQ ID NO: 2, amino acids about 28-179 of SEQ ID NO: 4, and amino acids about 26-206 of SEQ ID NO: 6.

3. The method of claim 2 wherein the type of mature hematopoietic cells are selected from the group consisting of red blood cells, granulocytes, monocytes and platelets.

4. The method of claim 2, wherein the mature hematopoietic cells are red blood cells.

5. A method of increasing hematocrit in a mammal in need thereof comprising administering a therapeutically effective amount of a polypeptide comprising an amino acid sequence selected from the group consisting of: SEQ ID NO: 2, SEQ ID NO: 4, SEQ ID NO: 6, amino acids about 25-177 of SEQ ID NO: 2, amino acids about 28-179 of SEQ ID NO: 4, and amino acids about 26-206 of SEQ ID NO: 6.

6. The method of any one of claims 1-5 further comprising administering a therapeutically effective amount of at least one additional hematopoietic cytokine, wherein the hematopoietic cytokine is administered prior to, simultaneously with, or subsequent to the polypeptide comprising an amino acid sequence selected from the group consisting of, SEQ ID NO: 2, SEQ ID NO: 4, SEQ ID NO: 6, amino acids about 25-177 of SEQ ID NO: 2, amino acids about 28-179 of SEQ ID NO: 4, and amino acids about 26-206 of SEQ ID NO: 6.

7. The method of claim 6 wherein the at least one additional hematopoietic cytokine is selected from the group consisting of: Epo, TPO, IL-1, IL-3, IL-4, IL-5, IL-7, IL-9, IL-11, G-CSF, GM-CSF, M-CSF and SCF.

8. A method of decreasing the number of one or more type(s) of hematopoietic progenitor cells in a mammal in need thereof comprising administering a therapeutically effective amount of an antibody against a polypeptide comprising an amino acid sequence selected from the group consisting of, SEQ ID NO: 2, SEQ ID NO: 4, SEQ ID NO: 6, amino acids about 25-177 of SEQ ID NO: 2, amino acids about 28-179 of SEQ ID NO: 4, and amino acids about 26-206 of SEQ ID NO: 6.

9. A method for treating or preventing a hematopoietic disorder in a mammal comprising the administration to said mammal in need of such treatment a pharmaceutical composition comprising a therapeutically effective amount of at least one polypeptide comprising an amino acid sequence selected from the group consisting of, SEQ ID NO: 2, SEQ ID NO: 4, SEQ ID NO: 6, amino acids about 25-177 of SEQ ID NO: 2, amino acids about 28-179 of SEQ ID NO: 4, and amino acids about 26-206 of SEQ ID NO: 6.

10. The method of claim 9 wherein the method further comprises administration to said mammal a pharmaceutical composition comprising a therapeutically effective amount of at least one additional hematopoietic cytokine.

11. The method of claim 10 wherein the hematopoietic cytokine is administered prior to, simultaneously with, or subsequent to the administration of a polypeptide comprising an amino acid sequence selected from the group consisting of, SEQ ID NO: 2, SEQ ID NO: 4, SEQ ID NO: 6, amino acids about 25-177 of SEQ ID NO: 2, amino acids about 28-179 of SEQ ID NO: 4, and amino acids about 26-206 of SEQ ID NO: 6.

12. The method of claim 11 wherein the hematopoietic cytokine is selected from the group consisting of Epo, TPO, IL-1, IL-3, IL-4, IL-5, IL-7, IL-9, IL-11, G-CSF, GM-CSF, M-CSF and SCF.

13. The method of claim 12 wherein the composition comprising the polypeptide comprising an amino acid sequence selected from the group consisting of, SEQ ID NO: 2, SEQ ID NO: 4, SEQ ID NO: 6, amino acids about 25-177 of SEQ ID NO: 2, amino acids about 28-179 of SEQ ID NO: 4, and amino acids about 26-206 of SEQ ID NO: 6, further comprises the additional hematopoietic cytokine.

14. A method for treating or preventing a hematopoietic disorder in a mammal comprising the administration to said mammal in need of such treatment a pharmaceutical composition comprising a therapeutically effective amount a polypeptide comprising an amino acid sequence selected from the group consisting of, SEQ ID NO: 2, SEQ ID NO: 4, SEQ ID NO: 6, amino acids about 25-177 of SEQ ID NO: 2, amino acids about 28-179 of SEQ ID NO: 4, and amino acids about 26-206 of SEQ ID NO: 6.

15. The method of claim 14 wherein the method further comprises administration to said mammal a pharmaceutical composition comprising a therapeutically effective amount of at least one additional hematopoietic cytokine.

16. The method of claim 15 wherein the hematopoietic cytokine is administered prior to, simultaneously with, or subsequent to the administration of the LP82 polypeptide.

17. The method of claim 14 wherein the additional hematopoietic cytokine is selected from the group consisting of Epo, TPO, IL-1, IL-3, IL-4, IL-5, IL-7, IL-9, IL-1, G-CSF, GM-CSF, M-CSF and SCF.

18. A pharmaceutical composition comprising a hematopoietic progenitor cell-stimulating amount of a polypeptide comprising an amino acid sequence selected from the group consisting of, SEQ ID NO: 2, SEQ ID NO: 4, SEQ ID NO: 6, amino acids about 25-177 of SEQ ID NO: 2, amino acids about 28-179 of SEQ ID NO: 4, and amino acids about 26-206 of SEQ ID NO: 6.and a pharmaceutically acceptable carrier, diluent or excipient.

19. The pharmaceutical composition as in claim 18 further comprising at least one additional hematopoietic cytokine.

20. The pharmaceutical composition as in claim 18, wherein the at least one additional hematopoietic cytokine is selected from the group consisting of Epo, TPO, IL-1, IL-3, IL-4, IL-5, IL-7, IL-9, IL-11, G-CSF, GM-CSF, M-CSF and SCF.
